(19) United States
(12) Patent Application Publication
(10) Pub. No.: US 2013/0252291 A1
(43) Pub. Date: Sep. 26, 2013

(54) Processing Of Oils and Fats

(71) Applicant: Per Munk Nielsen, Hilleroed (DK)

(72) Inventor: Per Munk Nielsen, Hilleroed (DK)

(73) Assignee: Novozymes A/S, Bagsvaerd (DK)

(21) Appl. No.: 13/876,049

(22) PCT Filed: Oct. 11, 2011
(86) PCT No.: PCT/EP2011/067739

§ 371 Date: Jun. 6, 2013

(30) Foreign Application Priority Data
Oct. 13, 2010 (EP) 10187451.9

(51) Int. Cl. C12P 7/64

(52) U.S. Cl. CPC C12P 7/6418
USPC 435/134

(57) ABSTRACT

The invention relates to processing of oils and fats comprising applying an immobilized lipolytic enzyme, wherein a liquid aqueous lipolytic enzymes preparation is added to the immobilized lipolytic enzyme between feedstock batches, and/or is added to the immobilized lipolytic enzyme step wise or continuously during feedstock processing.

### FIELD OF THE INVENTION

The present invention relates to enzymatic processing of oils and fats. The invention particularly relates to the use of immobilized enzymes and liquid enzymes in such processing.

### BACKGROUND OF THE INVENTION

Enzymatic processing of oils and fats for biodiesel is technically feasible. Lipases catalyze i.a. the transesterification of a triglyceride substrate with alcohols such as methanol (MeOH) and ethanol (EtOH) to form fatty acid alkyl esters such as fatty acid methyl esters (FAME) and fatty acid ethyl esters (FAEE) respectively. Biodiesel produced by enzymatic bioconversion is, compared with chemical conversion, more environmental friendly and therefore desirable. However, with very few exceptions, enzyme technology is not currently used in commercial scale biodiesel production.

Therefore, there is still a need to develop improved and more cost effective methods applying lipolytic enzymes immobilized on low-cost carriers for used in production of fatty acid esters.

### SUMMARY OF THE INVENTION

The invention relates to processing of oils and fats comprising applying an immobilized lipolytic enzyme, wherein a liquid aqueous lipolytic enzymes preparation is added to the immobilized lipolytic enzyme between reactant feedstock batches, and/or is added to the immobilized lipolytic enzyme step wise or continuously during processing of the reactant feedstock.

The inventor has surprisingly found that by adding small amounts of a liquid lipolytic enzyme, e.g. in an aqueous solution, the activity of a carrier immobilized lipolytic enzyme can be regenerated, i.e. maintained at a level which allows prolonged use of the immobilized lipolytic enzyme in processing of oils and fats e.g. for production of biodiesel. Thereby is it possible to extend the process life of an immobilized enzyme preparation and reduce the number of production stops due to replacement of depleted enzyme carrier. Furthermore, the cost of catalyst per ton of biodiesel produced is reduced.

In a first aspect the invention relates to a method of producing a fatty acid ester product comprising: a) contacting a reactant feedstock with at least one immobilized lipolytic enzyme on a carrier, to provide a reaction mixture comprising the fatty acid ester product, b) adding to the at least one immobilized lipolytic enzyme a liquid preparation comprising at least one lipolytic enzyme, and, c) separating the resulting reaction mixture from the immobilized lipolytic enzyme, wherein step b) may be performed before, during and/or after step a). In a preferred embodiment of the first aspect the fatty acid ester product is biodiesel.

In a second aspect the invention relates to a use of the method of the first aspect for production of biodiesel or fat hardstock, e.g. for margarine.

### DETAILED DESCRIPTION OF THE INVENTION

### Fatty Acid Ester Products

A preferred fatty acid ester product is a methyl or ethyl ester of a fatty acid. Such an ester is suitable for use as biodiesel. Biodiesel represents an alternative fuel for use in compression-ignition (diesel) engines. Herein the term biodiesel is used broadly for fatty acid alkyl esters of short-chain alcohols. A short-chain alcohol is an alcohol having 1 to 5 carbon atoms (C₁-C₅). A preferred short-chain alcohol is methanol or ethanol. Further preferred short-chain alcohols are propanol, isopropropanol and/or butanol.

While the method of the invention is especially contemplated for production of biodiesel, i.e. in an alcoholysis reaction, the method of the invention is equally applicable in processes such as degumming of edible oils or interesterification of fat hardstock for margarine.

### Lipolytic Enzymes

The term “lipolytic enzyme” is defined herein as an enzyme comprising one or more activity selected from triacylglycerol lipase activity, EC 3.1.1.3 i.e. hydrolytic activity for carboxylic ester bonds in triglycerides, and/or phospholipase activity (A1 or A2 or C, EC3.1.1.32 or 3.1.1.4 or 3.1.4.12), i.e. hydrolytic activity towards one or both carboxylic ester bonds in phospholipids such as lecithin, cutinase activity (EC 3.1.1.74), and acyltransferase activity (EC 2.3.1.43). Triacylglycerol lipase activity, EC 3.1.1.3 is suitable for use in biodiesel processes and for interesterification of fat hardstock for margarine. Phospholipase activity (A1 or A2 or C, EC 3.1.1.32 or 3.1.1.4 or 3.1.4.12) is suitable for degumming.

The lipolytic enzyme may catalyze reactions such as alcoholysis, esterification, interesterification, transesterefication, esterification, condensation and glycerolysis and aminolysis. Most lipolytic enzymes used as catalysts in organic synthesis are of microbial and fungal origin, and these are readily available by fermentation and basic purification.

In certain embodiments the present invention relates to a method of producing a fatty acid ester product , wherein the at least one immobilized lipolytic enzyme and/or the at least one lipolytic enzyme in the liquid preparation is at least 60%, at least 70%, at least 75%, at least 80%, at least 85%, at least 88%, at least 90%, at least 92%, at least 94%, at least 95%, at least 96%, at least 97%, at least 98%, or at least 99% identical to an enzyme selected from the group containing: Thermomyces lanuginosus lipase (Humicola lanuginosa) (positions 1-269 of SEQ ID NO: 2 of U.S. Pat. No. 5,869,438).; Candida Antarctica A lipase (SEQ ID NO:2 in WO 199401541); Candida Antarctica B lipase (UNIPROT: P41365); Candida deformans lipase; Candida lipolytica lipase; Candida parapsilosis lipase; Candida rugosa lipase; Cryptococcus spp. S-2 lipase; Rhizomucor miehei lipase (SEQ ID NO:4 in WO 199401541); Rhizomucor delemar lipase; Burkholderia (Pseudomonas) cepacia lipase; Pseudomonas camembertii lipase; Pseudomonas fluorescens lipase; Geotrichium candidum lipase (UNIPROT: P17573); Hyphozyma sp. lipase; Klebsiella oxytoca lipase, lipase/phospholipase from Fusarium oxysporum (SEQ ID NO:2 in WO 199826057), lysophospholipases from Aspergillus niger and A. oryzae (WO 200127251), phospholipase A1 from Aspergillus oryzae (EP 575133, JP-A 10-155493), lysophospholipase from F. venenatum (WO 200028044), phospholipase B from A. oryzae US 6146869), lipase from A. tubigensis (WO 199845453), lipase from F. solani (US 5,990.069), lipolytic enzyme from F. culmorum (U.S. Pat. No. 5,830,736), phospholipase from Hyphozyma (U.S. Pat. No. 6,127,137), an acyltransferase described in WO 2004064537, or WO 200506634 and a variant obtained by altering the amino acid sequence a lipolytic enzyme, e.g. one of the above, e.g. as described in WO 200032758, particularly Examples 4, 5, 6 and 13, such as variants of the lipase from Thermomyces lanuginosus (also called Humicola lanuginosa).

The same lipolytic enzyme may be applied as the at least one immobilized lipolytic enzyme and as the at least one lipolytic enzyme in the liquid preparation. Alternatively, the at least one immobilized lipolytic enzyme and as the at least one lipolytic enzyme in the liquid preparation may be two different lipolytic enzymes.

The identity may be calculated based on either amino acid sequences or nucleotide sequences.

The relatedness between two amino acid sequences or between two nucleotide sequences is described by the parameter “identity”. For purposes of the present invention, the degree of identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends in Genetics 16: 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

### Immobilization of Lipolytic Enzymes

The present invention relates to a method of producing a fatty acid ester product, wherein at least one lipolytic enzyme is immobilized either on a carrier; by entrapment in natural or synthetic matrices, such as hydrophobic polymers, ion exchanged resins, sol-gels, alginate, and carrageenan; by cross-linking methods such as in cross-linked enzyme crystals (CLEC) and cross-linked enzyme aggregates (CLEA); or by precipitation on salt crystals such as protein-coated micro-crystals (PCMC).

In certain embodiments the present invention relates to a method of producing a fatty acid ester product, wherein the carrier is a hydrophilic carrier selected from the group containing: porous in-organic particles composed of alumina, silica and silicates such as porous glas, zeolites, diatomaceous earth, bentonite, vermiculite, hydrotalcite; and porous organic particles composed of carbohydrate polymers such as agarose or cellulose. In other embodiments the present invention relates to a method of producing a fatty acid ester product, wherein the carrier is a hydrophobic polymeric carrier, e.g. polypropylen, polyethylene, acrylate. Suitable commercial carriers are e.g. Lewatit, Accurel, Purolit and Amberlite.

Two commonly applied commercial enzymes, NOVOZYM 435 and LIPOZYME TL IM represent examples of lipolytic enzymes immobilized on a hydrophobic carrier (NOVOZYM 435) and on a hydrophilic carrier (LIPOZYME TL IM), respectively.

### Liquid Lipolytic Enzyme Solution

The present invention relates to a method of producing a fatty acid ester product, wherein at least one lipolytic enzyme is in a liquid preparation, e.g. in an aqueous preparation. The preparation may comprise other constituents in addition to the at least one lipolytic enzyme. The at least one lipolytic enzyme may also be solubulized or suspended in one or more of the reactants and thus feed to the carrier during processing.

Preferably the at least one lipolytic enzyme in a liquid preparation is added to the carrier in an amount to maintain the activity at a level of at least 70%, at least 80%, at least 90%, or even at least 95% of the initial activity.

The at least one lipolytic enzyme in liquid preparation may comprise a different lipase or even a different type of lipolytic enzyme that the one used as immobilized enzyme as long as the reaction catalyzed by the liquid lipolytic preparation is desired in the reaction. The immobilized enzyme may comprise a lipase from C.antractica B (such as NOVOZYM 435 from Novozymes A/S) and the liquid preparation may comprise a lipase T. lanuginosus (example LIPOZYME TL 100L from Novozymes A/S). Alternatively, the liquid lipolytic preparation may comprise a lipolytic enzyme having phospholipase activity.

### Molar Ratio of Ethanol to Fatty Acid (EtOH:FA)

Excess of alcohol may drive the equilibrium reaction towards full conversion. For the purpose of the present invention the amount of alcohol is stated in equivalents (eq.) that is molar ratio of ethanol to fatty acid present in the substrate (EtOH:FA). The fatty acid may be esterified to glycerol or may be free fatty acid.

In certain embodiments the present invention relates to a method of producing a fatty acid ester product, wherein the molar ratio of ethanol to fatty acid in the substrate (EtOH:FA) is at least 0.2, 0.5, 0.75, 1.0, 1.1, 1.2, 1.3, 1.5, 2.0 equivalents.

Proteins are in general unstable in the presence of short-chain alcohols such as methanol and ethanol and inactivation of lipolytic enzymes occurs rapidly upon contact with insoluble alcohol, which exists as drops in the oil/ester phase. Accordingly, it may be desirable that the amount of alcohol is kept below its solubility limits in oil. This may be obtained by a continuous or step-wise addition of alcohol.

In certain embodiments the present invention relates to a method of producing a fatty acid ester product, wherein ethanol is added continuous or step-wise.

Depending on the total amount of ethanol to be used in the conversion reaction the number of steps in step-wise addition may vary. Thus, step-wise addition may constitute at least 2 steps; at least 3 steps; at least 4 steps; at least 5 steps; at least 6 steps; at least 7 steps; at least 8 steps; at least 9 steps; or at least 10 steps.

Conversion of a triglyceride-substrate results in formation of glycerol as a byproduct. Glycerol has been shown to inactivate immobilized enzymes, presumably by physically blocking the access of substrate to the enzyme. Steps of washing and drying may be included for the purpose of removing in particular glycerol which is considered to inhibit the activity of the lipolytic enzyme. A washing step may use at least one organic solvent, such as e.g. hexane and/or tert-butanol (t-BuOH). These solvents may be present during the reaction as part of the reaction mixture acting like a co-solvent for the alcohol.

### Enzymatic Biodiesel Process Design

In certain embodiments the present invention relates to a method of producing a fatty acid ester product, wherein said method is selected from the group of process designs consisting of: batch, continuous stirred-tank reactor, packed bed column, moving packed bed, and expanded bed reactor.

The batch process is a typical process used in the laboratory due to the simple setup. This process can be operated with addition of all components from the start, i.e., in bulk, or with step-wise addition of alcohol which is recommended. The batch process is useful in collecting data about the process, as for instance productivity of the enzyme.

A continuous stirred tank reactor is a container with a continuous supply of feed and withdrawal of product. The design applies multiple tanks in series to assure the same degree of conversion for the same reaction time, as well as a large total tank volume.

A system of packed bed columns with immobilized enzymes results in a well defined contact time between the liquid reactants and the solid catalyst. Furthermore, with this setup the enzyme to substrate ratio will be high at any specific time, and the whole system can be designed to be relatively compact. Commercial scale precedence for this technology already exists for enzymatic interesterification of oils.

### Reactant Feedstock

Fatty acid ethyl esters for biodiesel may be prepared from several types of vegetable oils. Examples of plants which may serve as reactant feedstock for use as substrate in the production of fatty acid ethyl esters are such as babassu, borage, canola, coconut, corn, cotton, hemp, jatropha, karanj, mustard, palm, peanut, rapeseed, rice, soybean, and sunflower.

Microalgae is also considered as feedstock in the production of biodiesel due to the higher photosynthetic efficiency of microalgae in comparison with plants and hence a potentially higher productivity per unit area.

Alternatively, fatty acid ethyl esters may be prepared from non-vegetable feedstocks like animal fat such as lard, tallow, butterfat and poultry; or marine oils such as tuna oil and hoki liver oil.

It has been estimated that 60-90% of the biodiesel cost arises from the cost of the feedstock oil, and thus use of cheaper waste oil would have a great impact in reducing the cost of biodiesel. In addition, it is considered an important step in reducing and recycling waste oil. Fresh vegetable oil and its waste differ in their content of water and free fatty acid. Unlike the conventional chemical routes for synthesis of diesel fuels, biocatalytic routes permit one to carry out the transesterification of a wide variety of oil feedstocks in the presence of acidic impurities, such as free fatty acids. Accordingly, fatty acid distillates (from deodorizer/fatty acid stripping), acid oils (from soap stocksplitting in chemical oil refining), waste oils and used oils may serve as feedstock in the production of biodiesel.

Thus, the feedstock can be of crude quality or further processed (refined, bleached and deodorized). Suitable oils and fats may be pure triglyceride or a mixture of triglyceride, diglyceride, monoglyceride, and free fatty acids, commonly seen in waste vegetable oil and animal fats. The feedstock may also be obtained from vegetable oil deodorizer distillates. The type of fatty acids in the feedstock comprises those naturally occurring as glycerides in vegetable and animal fats and oils. These include oleic acid, linoleic acid, linolenic acid, palmetic acid and lauric acid to name a few. Minor constituents in crude vegetable oils are typically phospholipids, free fatty acids and partial glycerides i.e. mono- and diglycerides.

In certain embodiments the present invention relates to a method of producing a fatty acid ester product, wherein the feedstock is selected from the group containing: babassu oil; borage oil; canola oil; coconut oil; corn oil; cotton oil; hemp oil; jatropha oil; karanj oil; mustard oil; palm oil; peanut oil; rapeseed oil; rice oil; soybean oil; and sunflower oil; oil from microalgae; animal fat; tallow; lard; butterfat; poultry; marine oils; tuna oil; hoki liver oil; fatty acid distillates; acid oils; waste oil; used oil; brown grease; yellow grease; partial glycerides and any combinations thereof.

For interesterification the feedstock comprises at least two fatty acids ester, e.g. a fatty acid ester with a high melting point, such as coconut oil, or palm oil, and a fatty acid ester with a low melting point, such as soybean oil, sun flower oil, rape seed oil or other oil having a melting point below room temperature (25° C.).

### Materials and Methods

### Lipolytic Activity

The lipolytic activity may be determined using tributyrine as substrate. This method is based on the hydrolysis of tributyrin by the enzyme, and the alkali consumption is registered as a function of time.

One Lipase Unit (LU) is defined as the amount of enzyme which, under standard conditions (i.e. at 30° C.; pH 7.0; with 0.1% w/v Gum Arabic as emulsifier and 0.16 M tributyrine as substrate) liberates 1 micromol titrable butyric acid per minute. One KLU is 1000 LU.

### Lipolytic Enzyme

LIPOZYME TL 100 L is a commercial preparation comprising a lipase from T. lanuginosus (amino acid sequence shown in positions 1-269 of SEQ ID NO: 2 of U.S. Pat. No. 5,869,438). LIPOZYME TL 100 L is available from Novozymes A/S.

### EXAMPLE 1

Fatty acid ethyl esters (FAEE) were synthesized using soybean oil and ethanol in a batch reaction catalyzed by lipase immobilized on Lewatit. The effect of addition of a liquid lipase after each batch was studied.

Two types of immobilized enzyme were used. Enzyme 1: LIPOZYME TL 100L was immobilized on Lewatit VPOC 1600 in an amount corresponding to 500 KLU/g carrier. This enzyme preparation was dried after immobilization. Enzyme 2: As Enzyme 1 but prepared just before the reaction without drying the enzyme before oil and ethanol are added.

Dosage of carrier with immobilized lipase was 2% w/w of oil soybean oil based on dry carrier weight.

LIPOZYME TL 100L was used as the liquid formulation for addition after each batch. Dosage of liquid lipase was 25 KLU/g carrier per batch corresponding to 0.25 g LIPOZYME TL 100L per g carrier. The first batch was processed without adding liquid lipase.

Azeotropic ethanol was used as the acyl-donor in an amount corresponding to 1.2 molar equivalents to the amounts of fatty acids in the soybean oil.

The reactions were performed in batch size of 10 g in 20 mL glass reactors. The reactors were mounted in a shaking cabinet which moved the reactors at a speed of 200 rpm at temperature 35° C.The ethanol was added continuously, i.e. 1.95 g-2.47 ml during 18 hours =0.137 ml/h.

Total reaction time was 20 hours. Fatty acid ethyl esters in oil phase were measured using gas chromatography (GC).

The addition of liquid lipase maintained the lipase activity in the system and resulted in increased production of fatty acid ethyl esters.

TABLE 1
Effect of addition of liquid lipase after each batch of soybean oil
and ethanol shown as percentage conversion into FAEE.

| | Enzyme 1 | Enzyme 1 with | Enzyme 2 | Enzyme 2 with |
|---|---|---|---|---|
| Batch # | control | 25 KLU added | control | 25 KLU added |
| 1 | 83.6 | 83.6 | 95.2 | 93.9 |
| 2 | 58.9 | 89.1 | 96.3 | 95.4 |
| 3 | 68.7 | 82.5 | 75.8 | 94.2 |
| 4 | 69.3 | 84.4 | 67.1 | 88.3 |
| 5 | 64.6 | 86.0 | 64.5 | 85.1 |
| 6 | 63.9 | 83.8 | 56.9 | 75.5 |

1-13. (canceled)

14. A method of producing a fatty acid ester product comprising:
a) contacting a reactant feedstock with at least one immobilized lipolytic enzyme on a carrier, to provide a reaction mixture comprising the fatty acid ester product,
b) adding to the at least one immobilized lipolytic enzyme a liquid preparation comprising at least one lipolytic enzyme, and
c) separating the resulting reaction mixture from the immobilized lipolytic enzyme, wherein step b) may be performed before, during and/or after step a).

15. The method of claim 14, wherein the reactant feedstock comprises at least one fatty acid ester, and an alcohol, preferably an alcohol selected from methanol or ethanol.

16. The method of claim 14, wherein the reactant feedstock comprises a first fatty acid ester, and a second fatty acid ester.

17. The method of claim 14, wherein the reactant feedstock comprises triglycerides, diglycerides, monoglycerides, free fatty acids, or any combination thereof.

18. The method of claim 14, wherein the at least one lipolytic enzyme is selected from lipase, cutinase, phospholipase (A1, A2, B, C, D), acyltransferase or any combination thereof.

19. The method of claim 14, wherein the process performed is selected from alcoholysis, esterification, interesterification, transesterification, condensation and glycerolysis.

20. The method of claim 14, wherein the fatty acid ester product comprises fatty acid alkyl esters, preferably methyl-, ethyl-, propyl-, isopropyl-, or butyl-esters.

21. The method of claim 14, wherein the at least one immobilized lipolytic enzyme is immobilized on a carrier by adsorption

22. The method of claim 14, wherein the at least one immobilized lipolytic enzyme is immobilized on a carrier by entrapment.

23. The method of claim 14, wherein the liquid preparation comprising at least one lipolytic enzyme further comprises water, glycerol, and/or a liquid reactant.

24. The method of claim 14, wherein the carrier is a hydrophilic carrier selected from the group consisting of: porous in-organic particles composed of alumina, silica and silicates such as porous glass, zeolites, diatomaceous earth, bentonite, vermiculite, hydrotalcite; and porous organic particles composed of carbohydrate polymers such as agarose or cellulose, or polymers such as polypropylene, polyethylene, and acrylate.

25. The method of claim 14, wherein said method is selected from the group of process designs consisting of: batch, continuous stirred tank reactor, packed bed reactor, moving packed bed reactor, and expanded bed reactor.

26. The method of claim 14, wherein the reactant feedstock is selected from the group consisting of: babassu oil; borage oil; canola oil; coconut oil; corn oil; cotton oil; hemp oil; jatropha oil; karanj oil; mustard oil; palm oil; peanut oil; rapeseed oil; rice oil; soybean oil;
and sunflower oil; oil from microalgae; animal fat; tallow; lard; butterfat; poultry; marine oils; tuna oil; hoki liver oil; fatty acid distillates; acid oils; waste oil; used cooking oil; partial glycerides and any combinations thereof.
